(19) United States
(12) Patent Application Publication
(10) Pub. No.: US 2015/0353959 A1
(43) Pub. Date: Dec. 10, 2015

(54) PEPTIDES FOR ENHANCING PROTEIN EXPRESSION

(71) Applicant: GLYCOTOPE GMBH, Berlin (DE)

(72) Inventors: Steffen GOLETZ, Berlin (DE); Antje DANIELCZYK, Berlin (DE); Doreen JAHN, (US)

(21) Appl. No.: 14/760,250

(22) PCT Filed: Jan. 17, 2014
(86) PCT No.: PCT/EP2014/050882

§ 371 Date: Jul. 10, 2015

(30) Foreign Application Priority Data
Jan. 18, 2013 (EP) 13151855.7

(51) Int. Cl. C12N 15/85; C07K 14/505; C07K 14/47

(52) U.S. Cl. CPC C12N 15/85; C07K 14/47; C07K 14/505; C07K 2319/35; C07K 2319/50

(57) ABSTRACT

The present invention pertains to the field of recombinant protein production. Novel peptides derived from the extracellular region of a glycophorin protein are provided which enhance the expression rate of proteins or peptides of interest when expressed as fusion protein together with said novel peptides.

### FIELD OF THE INVENTION

The present invention pertains to novel peptides which can be used to enhance the production yield of a protein of interest. The peptides are derived from the extracellular domain of a glycophorin protein and are used as part of a fusion protein with the protein of interest. The present invention in particular provides an expression cassette comprising such a peptide as part of the open reading frame.

### BACKGROUND OF THE INVENTION

Recombinant protein production is a major aspect of the biotechnical industry of today. It is gaining more and more importance as the number of applications requiring high amounts of high-quality proteins increase on the market. Food production and in particular pharmacology are two main areas where the need for recombinant proteins steadily increases. Higher production efficiencies and consequently lower costs of the final product are needed for obtaining a commercially viable process.

However, at the same time a high product quality and compatibility with human applications is essential. More and more applications required recombinant production of the proteins in eukaryotic cells, in particular in higher eukaryotic cells. Especially proteins carrying post-translational modifications such a glycosylation (glycoproteins) significantly differ when expressing them in prokaryotic cell systems such as E. coli or eukaryotic cell systems such as in particular human cell lines. These differences in many cases markedly affect the biological activity as well as the immunogenicity of the produced proteins. However, many expression systems using higher eukaryotic cell lines suffer from a rather low expression rate of the desired protein, resulting in low yields and high costs of the recombinant protein.

Therefore, there is a need in the art to provide novel means and methods for increasing the yield of recombinant protein production, especially when using eukaryotic expression cell lines.

### SUMMARY OF THE INVENTION

The present inventors could demonstrate that the yield and production rate of different proteins is markedly increased when they are expressed as fusion protein fused to the extracellular region or a part thereof of a glycophorin protein. Compared to the expression of the protein of interest alone under the same conditions using the same expression vector, the expression rate of the fusion protein is increased up to 10-fold. For subsequent applications of the protein of interest, it can be used as fusion protein or the extracellular region or part thereof of the glycophorin protein can be cleaved off from the protein of interest using a protease recognition site constructed between the two fusion partners.

In view of the above, the present invention provides in a first aspect an expression cassette comprising a promoter region, an expression element and optionally a transcription terminator region, wherein the expression element comprises a nucleic acid sequence coding for the extracellular region or a part thereof of a glycophorin protein, and wherein the expression cassette does not comprise a nucleic acid sequence coding for the entire glycophorin protein. In particular the expression element of the expression cassette further comprises a nucleic acid sequence coding for a peptide of interest, wherein the peptide of interest and the extracellular region or part thereof of the glycophorin protein form a fusion peptide when expressing the expression element.

In further aspects, the present invention provides a vector comprising the expression cassette according to the first aspect and a host cell comprising said expression cassette or said vector.

In another aspect, the present invention provides a method for producing a peptide of interest, comprising the steps of
(a) providing a host cell comprising the expression cassette according to the first aspect including a nucleic acid sequence coding for the peptide of interest;
(b) culturing the host cell under conditions at which the host cell expresses a fusion peptide comprising the peptide of interest and the extracellular region or part thereof of the glycophorin protein;
(c) obtaining the peptide of interest, optionally in the form of said fusion peptide.

Furthermore, the present invention provides a fusion peptide comprising the extracellular region or a part thereof of a glycophorin protein and a peptide of interest, obtainable by the method for producing a peptide of interest according to the invention.

In another aspect, the present invention provides a method for increasing the yield of a peptide of interest in recombinant production, comprising the step of expressing the peptide of interest as part of a fusion peptide which further comprises the extracellular region or a part thereof of a glycophorin protein. Furthermore, also the use of the extracellular region or a part thereof of a glycophorin protein in a fusion peptide together with a peptide of interest for increasing the yield of said peptide of interest in recombinant production is provided by the present invention.

The above aspects can be combined. Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### DEFINITIONS

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”.

An “expression cassette” is a nucleic acid construct, generated or synthetically, with nucleic acid elements that are capable of effecting expression of a structural gene in hosts that are compatible with such sequences. Expression cassettes include at least promoters and optionally, transcription termination signals. Typically, the expression cassette includes a nucleic acid to be transcribed and a promoter. Additional factors helpful in effecting expression may also be used as described herein. For example, an expression cassette can also include nucleotide sequences that encode a signal sequence that directs secretion of an expressed protein from the host cell. An expression cassette preferably is part of an expression vector. Host cells which shall be used for expression of the nucleic acid to be transcribed are transformed or transfected with the expression vector. To allow selection of transformed cells comprising the constructs, a selectable marker gene can be conveniently included in the expression vectors. A person having skill in the art will recognize that this vector component can be modified without substantially affecting its function.

The expression “functionally linked to one another” means that said elements of an expression cassette are linked to one another in such a way that their function is coordinated and allows expression of the coding sequence (e.g. the expression element). By way of example, a promoter is functionally linked to a coding sequence when it is capable of ensuring expression of said coding sequence. The construction of an expression cassette according to the invention and the assembly of its various elements can be carried out using techniques well known to those skilled in the art, in particular those described in Sambrook et al. (1989, Molecular Cloning: A Laboratory Manual, Nolan C. ed., New York: Cold Spring Harbor Laboratory Press).

A “homologue” of a target nucleic acid sequence or amino acid sequence shares a homology or identity of at least 75%, more preferably at least 80%, at least 85%, at least 90%, at least 93%, at least 95% or at least 97% with said target nucleic acid sequence or amino acid sequence. A “homology” or “identity” of an amino acid sequence or nucleotide sequence is preferably determined according to the invention over the entire length of the target sequence or over the entire length of the indicated part of the target sequence.

A “peptide” as used herein refers to a polypeptide chain comprising at least 5 amino acids. A peptide preferably comprises at least 10, at least 15, at least 20, at least 25, at least 30 or at least 35 amino acids. The term “peptide” as used herein also refers to proteins, including peptides and proteins which were post-translationally modified. In particular, the term peptide includes glycosylated peptides and glycoproteins.

A part of a peptide or protein preferably comprises at least 3 consecutive amino acids of said peptide or protein, preferably at least 5, at least 10, at least 15 or at least 20 consecutive amino acids of said protein.

The term “pharmaceutical composition” and similar terms particularly refers to a composition suitable for administering to a human, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

### DETAILED DESCRIPTION OF THE INVENTION

The present invention is directed to expression cassettes comprising a promoter region, an expression element and optionally a transcription terminator region, wherein the expression element comprises a nucleic acid sequence coding for the extracellular region or a part thereof of a glycophorin protein. The expression cassette is designed for expressing peptides of interest in host cells. A nucleic acid sequence coding for the peptide of interest may already be present in the expression element of the expression cassette according to the invention or may be cloned into said expression element. The peptide of interest and the extracellular region or part thereof of the glycophorin protein are functionally linked to one another in the expression element so that they are expressed as a fusion peptide.

The peptide of interest, however, is not the remaining part of the glycophorin protein and the expression cassette does not comprise a nucleic acid sequence coding for the entire glycophorin protein. In particular, the expression cassette does not comprise a nucleic acid sequence encoding for the transmembrane region and/or the cytoplasmatic region of the glycophorin protein.

The extracellular region or part thereof of the glycophorin protein

The glycophorin protein may be any member of the glycophorin protein family. Preferably, the glycophorin protein is a mammalian or rodent glycophorin protein, more preferably a human glycophorin protein. The glycophorin protein is in particular selected from the group consisting of glycophorin A, glycophorin B, glycophorin C and glycophorin E, and preferably is glycophorin A. In particularly preferred embodiments, the glycophorin protein is human glycophorin A having the amino acid sequence of SEQ ID NO: 1. Human glycophorin A is also known as MN sialoglycoprotein, PAS-2, sialoglycoprotein alpha and CD235a.

A glycophorin protein is composed of an N-terminal extracellular region, a transmembrane region and a C-terminal cytoplasmatic region. Furthermore, the glycophorin protein is expressed with an N-terminal localization signal peptide which is cleaved of after expression, thereby forming the mature glycophorin protein. Except when indicated otherwise, the term “glycophorin protein” as used herein refers to a mature glycophorin protein not comprising a localization signal peptide. The mature glycophorin protein does not comprise said localization signal peptide. This localization signal peptide does not form part of the extracellular region of the glycophorin protein. The extracellular region of a glycophorin protein therefore begins at the N-terminus of the mature glycophorin protein and ends at the amino acid positioned directly N-terminal of the transmembrane region. Transmembrane regions of any protein, in particular of any glycophorin protein, can be readily identified by the skilled person, for example using suitable sequence analysis tools. For known glycophorin proteins, the transmembrane domain is also indicated in commonly available protein databases (see, for example, the UniProtKB protein knowledgebase of the UniProt consortium, www.uniprot.org). The transmembrane domain of the human glycophorin A, for example, consists of amino acids 73 to 95 of SEQ ID NO: 1. Hence, the extracellular region of the human glycophorin A consists of amino acids 1 to 72 of SEQ ID NO: 1.

The part of the extracellular region of a glycophorin protein as used herein refers to a peptide comprising at least 10 consecutive amino acids of the extracellular region of a glycophorin protein as defined herein. Preferably, the part of the extracellular region comprising at least 15, more preferably at least 20, at least 25, at least 30 or at least 35 consecutive amino acids of the extracellular region of a glycophorin protein. In preferred embodiments, the part of the extracellular region includes amino acids located in the N-terminal region of the extracellular region. In particular, the part of the extracellular region comprises at least 10, preferably at least 15, more preferably at least 20, at least 25, at least 30 or at least 35 consecutive amino acids of the first 40 amino acids of the extracellular region. In case of the human glycophorin A, this means that the part of the extracellular region of said human glycophorin A comprises at least 10, preferably at least 15, more preferably at least 20, at least 25, at least 30 or at least 35 consecutive amino acids of the amino acid sequence of position 1 to 40 of SEQ ID NO: 1.

In certain embodiments, the term “extracellular region of a glycophorin protein” also encompasses homologues thereof. Said homologues have at least 70% amino acid sequence identity to the extracellular region of the glycophorin protein. Preferably, the sequence identity is at least 75%, more preferably at least 80%, at least 85%, at least 90% or at least 95%. The sequence identity is determined over the entire extracellular region of the glycophorin protein. In specific embodiments, the extracellular region of the glycophorin protein also includes homologues of the extracellular region of human glycophorin A, wherein said homologues have at least 70%, preferably at least 75%, more preferably at least 80%, at least 85%, at least 90% or at least 95% sequence identity to the amino acids sequence of position 1 to 72 of SEQ ID NO: 1. Likewise, a part of the extracellular region of a glycophorin protein in certain embodiments also refers to a part of a homologue of the extracellular region of a glycophorin protein. In preferred embodiments, the sequence identity between the homologue and the extracellular region as described above also applies to the part of the extracellular region. Hence, the part of a homologue of the extracellular region has at least 70%, preferably at least 75%, more preferably at least 80%, at least 85%, at least 90% or at least 95% amino acid sequence identity to the corresponding part of the extracellular region of the glycophorin protein.

In particularly preferred embodiments, the extracellular region or part thereof of the glycophorin protein comprises and in particular consists of amino acids 1 to 38 of SEQ ID NO: 1 or amino acids 2 to 38 of SEQ ID NO: 1.

The extracellular region or part thereof of the glycophorin protein preferably is capable of increasing the production rate and yield of a peptide of interest if said peptide of interest is expressed as fusion peptide together with the extracellular region or part thereof of the glycophorin protein, in particular in the host cells described herein.

The Expression Element

The expression element of the expression cassette according to the present invention comprises the nucleic acid sequences which are to be expressed by the expression cassette. The expression of the nucleic acid sequences of the expression element is regulated by the promoter region. When the expression cassette, optionally present in a vector, is introduced into a suitable host cell, said host cell produces a peptide coded for by the nucleic acid sequences of the expression element.

The expression element of the expression cassette according to the present invention preferably contains a cloning site or a nucleic acid sequence coding for a peptide of interest. The cloning site or the nucleic acid sequence coding for a peptide of interest may be located, in the direction of transcription, in front of or behind the nucleic acid sequence coding for the extracellular region or part thereof of the glycophorin protein. The nucleic acid sequence coding for the peptide of interest is present in the expression element in frame with the nucleic acid sequence coding for the extracellular region or part thereof of the glycophorin protein. The three-nucleotide frame of the nucleic acid sequence coding for the protein of interest is the same as that of the nucleic acid sequence coding for the extracellular region or part thereof of the glycophorin protein. During translation of these nucleic acid sequences, one polypeptide chain is produced comprising the peptide of interest and the extracellular region or part thereof of the glycophorin protein. The peptide of interest and the extracellular region or part thereof of the glycophorin protein form a fusion peptide when expressing the expression element. In the fusion peptide, the extracellular region or part thereof of the glycophorin protein may be located N-terminally or C-terminally of the peptide of interest.

The peptide of interest may be any peptide, including proteins. The peptide may be of any origin, including mammalian- and human-derived peptides as well as artificial peptides. Preferably, the peptide comprises one or more glycosylation sites and in particular is a glycosylated peptide such as a glycoprotein.

The cloning site present in the expression element is suitable for introducing a nucleic acid coding for a peptide of interest into the expression element. The cloning site is in particular designed to enable the introduction of a nucleic acid coding for a peptide of interest in such a manner that the nucleic acid sequence coding for the peptide of interest and the nucleic acid sequence coding for the extracellular region or part thereof of the glycophorin protein are in frame as described above. Suitable cloning sites and methods for introducing nucleic acid fragments into other nucleic acid molecules such as expression cassettes or vectors are commonly known in the art. The cloning site preferably comprises at least one, preferably at least two, at least three, at least four or at least five recognition sequences of restriction enzymes. Suitable restriction enzymes and their recognition sequences are known in the art. Exemplary restriction enzymes are EcoRI, EcoRV, HindIII, BamHI, XbaI and PvuI.

In certain embodiments, the expression element further comprises a nucleic acid sequence coding for a signal peptide which preferably comprises an extracellular localization signal. The signal peptide in particular induces a secretory expression of the fusion peptide encoded by the expression element. The signal peptide preferably is cleaved off from the remaining fusion peptide during the expression. The signal peptide preferably is positioned, in the direction of transcription, in front of the other coding nucleic acid sequences comprised in the expression element, in particular at the beginning of the expression element. Furthermore, the nucleic acid sequence coding for the signal peptide is preferably positioned in frame with the other coding nucleic acid sequences comprised in the expression element.

In further embodiments, the expression element further comprises a nucleic acid sequence coding for a protease recognition site. The nucleic acid sequence coding for the protease recognition site preferably is positioned between
(i) the nucleic acid sequence coding for the extracellular region or part thereof of the glycophorin protein and
(ii) the cloning site or the nucleic acid sequence coding for a peptide of interest.

The nucleic acid sequence coding for the protease recognition site is positioned in frame with the nucleic acid sequence coding for the extracellular region or part thereof of the glycophorin protein and optionally also with the nucleic acid sequence coding for the peptide of interest, as described above. The extracellular region or part thereof of the glycophorin protein, the protease recognition site and optionally the peptide of interest form a fusion peptide when expressing the expression element. When contacting such a fusion peptide with the respective protease, the protease cleaves the fusion peptide at the recognition site, forming one part of the fusion peptide comprising the peptide of interest and another part of the fusion peptide comprising the extracellular region or part thereof of the glycophorin protein. Suitable proteases and their recognition sites are known in the art. Preferably, the protease is an endopeptidase. Exemplary proteases are thrombin and factor Xa.

The nucleic acid sequences comprised in the expression element are preferably functionally linked to each other in the direction of transcription in the following order:
(i) optionally the nucleic acid sequence coding for a signal peptide;
(ii) the cloning site or a nucleic acid sequence coding for a peptide of interest;
(iii) optionally the nucleic acid sequence coding for a protease recognition site;
(iv) the nucleic acid sequence coding for the extracellular region or part thereof of the glycophorin protein.

Alternatively, the nucleic acid sequences comprised in the expression element are preferably functionally linked to each other in the direction of transcription in the following order:
(i) optionally the nucleic acid sequence coding for a signal peptide;
(ii) the nucleic acid sequence coding for the extracellular region or part thereof of the glycophorin protein.
(iii) optionally the nucleic acid sequence coding for a protease recognition site;
(iv) the cloning site or a nucleic acid sequence coding for a peptide of interest.

The elements (i) to (iv), if present, preferably form a fusion peptide when expressing the expression element.

The elements of the expression element, in particular the nucleic acid sequence coding for the signal peptide, the nucleic acid sequence coding for the extracellular region or part thereof of the glycophorin protein, the nucleic acid sequence coding for the protease recognition site and the nucleic acid sequence coding for the peptide of interest all form together one open reading frame. That means in particular that these elements are all in the same coding frame with each other and that there is no stop codon between said elements in said coding frame. Preferably, the first codon of the expression element is a start codon coding for methionine and the last codon of the expression element is a stop codon.

In particularly preferred embodiments, the expression element comprises the nucleic acid sequence coding for a peptide of interest.

The Further Elements of the Expression Cassette

In addition to the expression element, the expression cassette according to the invention further comprises at least a promoter region which is capable of initiating transcription of the expression element. The promoter region includes an RNA polymerase binding site, a transcription start site and transcription factor binding sites.

The promoter may further optionally comprise regulatory elements which regulate transcription of the expression element. Preferably, the promoter is selected from the group consisting of an SV40 promoter, a CMV promoter, an EF-1α promoter, a RSV promoter, a BROADS promoter, a murine rosa 26 promoter, a pCEFL promoter and a β-actin promoter.

Furthermore, the expression cassette according to the invention preferably comprises a transcription terminator region which is a section of genetic sequence that marks the end of a gene for transcription. The transcription terminator region in particular stops the RNA polymerase and causes it to dissociate from the DNA strand.

In preferred embodiments, the expression cassette according to the present invention further comprises one or more elements selected from the group consisting of a 5′ enhancer region, a 5′ untranslated region, a 3′ untranslated region, a polyadenylation signal and a 3′ enhancer region. In particular, the expression cassette according to the invention comprises, in the direction of transcription, functionally linked to each other,
(i) optionally a 5′ enhancer region,
(ii) a promoter region,
(iii) optionally a 5′ untranslated region,
(iv) an expression element,
(v) optionally a 3′ untranslated region,
(vi) optionally a polyadenylation site,
(vii) optionally a transcription terminator region, and
(viii) optionally a 3′ enhancer region.

The enhancer regions are short regions of DNA that can enhance the transcription level of the expression element. The enhancer regions can be bound by activator proteins (trans-acting factors) which recruit the RNA polymerase and the general transcription factors which then begin transcribing the gene. Enhancer regions may be located upstream (5′ enhancer region) or downstream (3′ enhancer region) of the promoter-expression element complex.

The 5′ untranslated region (5′ UTR) is located between the promoter region and the expression element and preferably contains elements for controlling gene expression by way of regulatory elements. For example, the 5′ UTR may comprise sequences that promote or inhibit translation initiation and binding sites for proteins that may affect the mRNA's stability or translation.

The 3′ untranslated region (3′ UTR) is located between the expression element and the polyadenylation site and may contain binding sites for proteins that may affect the mRNA's stability or location in the cell. Both 5′ UTR and 3′ UTR are transcribed together with the expression element and form part of the produced mRNA.

The polyadenylation site is a sequence motive that initiates the synthesis of a poly(A) tail to the transcribed RNA. The polyadenylation site may be located at the end of or inside the 3′ UTR.

The expression cassette preferably is adapted for expression in eukaryotic cells, preferably mammalian cells, more preferably human cells.

The Vector Comprising the Expression Cassette

In one aspect, the present invention pertains to a vector comprising the expression cassette according to the invention. The vector may be any vector suitable for transferring the expression cassette into a host cell. Respective vectors are known in the art. In particular, the vector is adapted for transfer into eukaryotic cells, preferably mammalian cells, more preferably human cells.

In addition to the expression cassette, the vector according to the invention may comprise further elements. For example, the vector may comprise one or more selection markers. Preferably, at least one of the selection markers is suitable for selecting host cells, in particular eukaryotic host cells, preferably mammalian host cells, more preferably human host cells, comprising the vector against host cells not comprising the vector. Suitable examples of the selection markers are genes which provide resistance against an antibiotic compound. Furthermore, the vector may comprise elements suitable for amplificating it in a prokaryotic host cell such as E. coli cells. Such elements for example include an origin of replication such as Col E1 Ori and a prokaryotic selection marker such as a gene providing resistance against a bactericide, e.g. ampicillin.

Preferably, the vector is a circular or linear double-stranded DNA, in particular a circular double-stranded DNA.

In certain preferred embodiments, the vector comprises the expression cassette with the expression element comprising a nucleic acid sequence coding for a peptide of interest.

The Host Cell Comprising the Expression Cassette or the Vector

In a further aspect, the present invention provides a host cell comprising the expression cassette according to the invention or the vector according to the invention. The host cell may be any cell suitable for transfection with the expression cassette or vector and in particular suitable for production of the peptide of interest. Preferably, the host cell is derived from an established expression cell line. The host cell preferably is a eukaryotic cell, more preferably a mammalian cell, most preferably a human cell. In preferred embodiments, the host cell is derived from human myeloid leukaemia cells. Specific examples of host cells are K562, NM-F9, NM-D4, NM-H9D8, NM-H9D8-E6, NM H9D8-E6Q12, GT-2X, GT-5s and cells derived from anyone of said host cells. K562 is a human myeloid leukemia cell line present in the American Type Culture Collection (ATCC CCL-243). The remaining cell lines are derived from K562 cells and have been selected for specific glycosylation features. Cell lines derived from K562 can be cultivated and maintained under the well known conditions suitable for K562. All these cell lines except for K562 cells were deposited according to the Budapest treaty. Information on the deposition can be found at the end of the specification.

Exemplary host cells are also described, for example, in WO 2008/028686. In preferred embodiments, the host cell is optimized for expression of glycoproteins having a specific glycosylation pattern. Preferably, the codon usage in the expression element and/or the promoter and the further elements of the expression cassette or vector are compatible with and, more preferably, optimized for the type of host cell used.

In certain embodiments, the host cell is an isolated host cell. Preferably, the host cell is not present in the human or animal body.

In certain preferred embodiments, the host cell is transfected with the vector which comprises the expression cassette with the expression element comprising a nucleic acid sequence coding for a peptide of interest.

The Production Method

The present invention provides a method for producing a peptide of interest, comprising the steps of
(a) providing a host cell according to the invention;
(b) culturing the host cell under conditions at which the host cell expresses a fusion peptide comprising the peptide of interest and the extracellular region or part thereof of the glycophorin protein;
(c) obtaining the peptide of interest, optionally in the form of said fusion peptide.

The host cell used in the method in particular is transfected with the vector or expression cassette according to the invention, wherein the expression element thereof comprises a nucleic acid sequence coding for the peptide of interest. Suitable conditions for culturing the host cells and expressing the fusion protein depend on the specific host cell, vector and expression cassette used in the method. The skilled person can readily determine suitable conditions and they are also already known in the art for a plurality of host cells. In preferred embodiments, the fusion peptide is secreted by the host cells into the culture medium. In these embodiments, the expression element preferably comprises a nucleic acid sequence coding for a signal peptide which comprises an extracellular localization signal.

The method for producing a peptide of interest preferably further comprises the step of
(d) isolating the peptide of interest, optionally in the form of said fusion peptide.

Step (d) preferably is performed between step (b) and step (c).

Isolation of the peptide of interest in particular refers to the separation of the peptide of interest from the remaining components of the cell culture. For example, in case the peptide of interest is secreted by the host cell, isolation of the peptide of interest includes the separation of the cell culture medium from the host cells, for example by centrifugation, and the separation of the peptide of interest from some or most of the components of the cell culture medium, for example by chromatographic methods. Suitable methods and means for isolating the peptide of interest are known in the art and can be readily applied by the skilled person.

In preferred embodiments, the host cell used in the method according to the invention is transfected with the vector or expression cassette according to the invention, wherein the expression element thereof comprises a nucleic acid sequence coding for the peptide of interest and a nucleic acid sequence coding for a protease recognition site which is positioned between the nucleic acid sequence coding for the extracellular region or part thereof of the glycophorin protein and the nucleic acid sequence coding for the peptide of interest. In these embodiments, the method preferably further comprises the steps of
(e) treating the fusion peptide with a protease capable of recognizing the protease recognition site, wherein the fusion peptide is cleaved into a first part being the peptide of interest and a second part comprising the extracellular region or part thereof of the glycophorin protein; and
(f) separating the peptide of interest from the second part of the fusion peptide.

Steps (e) and (f) are preferably performed between step (b) and step (c), more preferably between step (d) and step (c). The peptide of interest in these embodiments is preferably obtained as single peptide and not as fusion peptide.

Suitable proteases are described herein above. Conditions which are suitable for the protease digestion depend on the specific protease used and are known in the art. Separation of the peptide of interest from the second part of the fusion peptide can be performed by known methods, for example by chromatographic methods or size exclusion filtration.

In further embodiments, the method for producing a peptide of interest preferably further comprises the step of
(g) formulating the peptide of interest, optionally in the form of said fusion peptide, as a pharmaceutical composition.

Step (g) preferably is performed after step (c), in particular as last step of the method.

Formulating the peptide of interest as a pharmaceutical composition preferably comprises exchanging the buffer solution or buffer solution components of the composition comprising the peptide of interest. Furthermore, the formulation step may include lyophilization of the peptide of interest. In particular, the peptide of interest is transferred into a composition only comprising pharmaceutically acceptable ingredients.

The present invention further provides a fusion peptide comprising the extracellular region or a part thereof of a glycophorin protein and a peptide of interest, obtainable by the method according to the invention. Furthermore, the present invention provides a pharmaceutical composition comprising a fusion peptide comprising the extracellular region or a part thereof of a glycophorin protein and a peptide of interest, obtainable by the method according to the invention.

Increase in Production Yield

In a further aspect, the present invention provides a method for increasing the yield of a peptide of interest in recombinant production, comprising the step of expressing the peptide of interest as part of a fusion peptide which further comprises the extracellular region or a part thereof of a glycophorin protein. For expression of the peptide of interest as such a fusion peptide, the expression cassette, vector, host cell and/or production method according to the present invention are preferably used.

Furthermore, the present invention provides the use of the extracellular region or a part thereof of a glycophorin protein in a fusion peptide together with a peptide of interest for increasing the yield of said peptide of interest in recombinant production. For said recombinant production of the peptide of interest as such a fusion peptide, the expression cassette, vector, host cell and/or production method according to the present invention are preferably used.

All the embodiments and features described above also likewise apply to the methods and uses according to the invention.

Numeric ranges described herein are inclusive of the numbers defining the range. The headings provided herein are not limitations of the various aspects or embodiments of this invention which can be read by reference to the specification as a whole. According to one embodiment, subject matter described herein as comprising certain steps in the case of methods or as comprising certain ingredients in the case of compositions refers to subject matter consisting of the respective steps or ingredients. It is preferred to select and combine preferred aspects and embodiments described herein and the specific subject-matter arising from a respective combination of preferred embodiments also belongs to the present disclosure.

### EXAMPLES

### Example 1

### Comparison of the Expression of Erythropoietin Alone or as Fusion Protein

In this assay, the effect of a fusion with a part of the glycophorin A extracellular region on the expression of a model protein, human erythropoietin (EPO), was tested. A nucleic acid sequence coding for EPO was cloned into a standard vector for eukaryotic expression comprising an EF-1α promoter and a dihydrofolate reductase (DHFR) gene as selection marker. A leader sequence (extracellular expression signal) of IgK or GM-CSF was present for secreted expression of the EPO. Furthermore, a nucleic acid sequence encoding amino acids 1 to 38 of the mature human glycophorin A (GYPA ex.reg.) was cloned in frame into the vector, either between the extracellular expression signal and the EPO or behind the EPO. In a control vector, the glycophorin construct was not inserted and only the extracellular expression signal and the EPO were encoded. The constructs used in the assay were as follows:

| | |
|---|---|
| Control1: | IgK leader-EPO |
| Control2: | GM-CSF leader-EPO |
| ETag1.1: | IgK-leader-EPO-GYPA ex.reg. |
| ETag1.2: | IgK-leader-GYPA ex.reg.-EPO |
| ETag1.3: | GM-CSF-leader-GYPA ex.reg.-EPO |

The different vectors were each transfected into two different human leukemia-derived host cell lines. The cells were selected for positive transfectants using methotrexate and the resulting transfected cells were screened for cell growth and EPO productivity. The production rate of these cells in picogram EPO per cell per day (pcd) was determined for two different concentrations of the selection agent methotrexate. Cells with the Control2 construct were not further analyzed because their EPO productivity was very low in the initial screens. The maximum production rates of the different expression cells are summarized in the following table.

TABLE 1

| | |
|---|---|
| Expression | Production Rate [pcd] |

| | | | |
|---|---|---|---|
| Construct | Tag Position | NM-F9 cells | NM-H9D8 cells |

100 nM methotrexate

| | | | |
|---|---|---|---|
| Control1 | — | 0.06-0.7 | 0-3.9 |
| ETag1.1 | C-term. | 0-2.2 | 0.1-8.3 |
| ETag1.2 | N-term. | 0-2.3 | 0-0.44 |
| ETag1.3 | N-term. | 0-2.9 | 0-5.4 |

200 nM methotrexate

| | | | |
|---|---|---|---|
| Control1 | — | 0.04-0.18 | 0.1-0.6 |
| ETag1.1 | C-term. | 1.9-6.7 | / |
| ETag1.2 | N-term. | 1.2-4.9 | 6.8-14.8 |
| ETag1.3 | N-term. | 0.56-6.8 | 6.1 |

The data demonstrate that the introduction of the extracellular region fragment of glycophorin A significantly increases the production rate of the protein of interest, i.e. EPO. This increase in production rate is independent of the position of the glycophorin fragment. N-terminal and C-terminal fusions provide comparable high production rates. Even when disregarding the different methotrexate concentrations used for the two setups, nearly 10-fold increases in the production rate are obtained using the glycophorin fragment fusion constructs. Within the 200 nM methotrexate concentration group, i.e. with a higher selection pressure for transfected cells with multiple copies of the vector, increases of up to 20-fold to nearly 40-fold were demonstrated. Furthermore, the fusion constructs according to the invention also show increased production rates independent of the expression cell line used.

### Example 2

### Comparison of the Expression of Factor VII Alone or as Fusion Protein

The effect of the glycophorin extracellular region fragment on the production of the further target protein factor VII was also analyzed. The constructs were designed and the assay was performed as described in Example 1, except that the glycophorin extracellular region tag consisted of amino acids 2 to 38 of the mature human glycophorin A. As signal peptide, the leader sequence of factor VII was used. As further control, a hemoglobin tag (HB-Tag) was N-terminally fused to factor VII. The following constructs were used:

| | |
|---|---|
| Control1: | leader-factor VII |
| Control2: | leader-HB-Tag-factor VII |
| ETag2.1: | leader-GYPA ex.reg.-factor VII |
| ETag2.2: | leader-factor VII-GYPA ex.reg. |

The mean production rates obtained in this assay are listed in the following table:

TABLE 2

| | | |
|---|---|---|
| | Expression | Mean Production Rate [pcd] |
| Construct | Tag Position | NM-H9D8 cells |

100 nM methotrexate

| | | |
|---|---|---|
| Control1 | — | 3.90 |
| Control2 | N-term. | 0.77 |
| ETag2.1 | N-term. | 6.14 |
| ETag2.2 | C-term. | 4.71 |

200 nM methotrexate

| | | |
|---|---|---|
| Control1 | — | 4.00 |
| Control2 | N-term. | 0.14 |
| ETag2.1 | N-term. | 6.65 |
| ETag2.2 | C-term. | 5.55 |

The data again demonstrate that the introduction of the extracellular region fragment of glycophorin A significantly increases the production rate of different proteins of interest. The mean production rate of factor VII is increased by up to 65% compared to the control construct. Again, the production rate is increased for both constructs regardless of the position of the glycophorin fragment. N-terminal and C-terminal fusions provide comparable high production rates. A control fusion tag derived from the hemoglobin protein, however, did not show increased production but rather resulted in a greatly reduced production rate.

It is to be noted that for factor VII the mean production rates of all picked clones was determined, while for EPO (Example 1) the range of different production rates of the individual clones is indicated. The much higher increase in production yield for EPO mentioned above hence refers to the maximum possible production rates obtained with the different constructs while for factor VII, only the mean production rates of all clones were compared. Since for further production processes the clone with the highest productivity is chosen, a comparison of said highest producing clones more likely reflects the actual increase in production yield.

Identification of the Deposited Biological Material

The cell lines DSM ACC 2606 and DSM ACC 2605 were deposited at the DSMZ—Deutsche Sammlung von Mikroorganismen und Zellkulturen GmbH, Mascheroder Weg 1b, 38124 Braunschweig (DE) by Nemod Biotherapeutics GmbH & Co. KG, Robert-Rössle-Str. 10, 13125 Berlin (DE). Glycotope is entitled to refer to these biological materials since they were in the meantime assigned from Nemod Biotherapeutics GmbH & Co. KG to Glycotope GmbH.

The cell lines DSM ACC 2806, DSM ACC 2807, DSM ACC 2856, DSM ACC 2858 and DSM ACC 3078 were deposited at the DSMZ—Deutsche Sammlung von Mikroorganismen und Zellkulturen GmbH, Inhoffenstraβe 7B, 38124 Braunschweig (DE) by Glycotope GmbH, Robert-Rössle-Str. 10, 13125 Berlin (DE).

| Name of the | Accession | | Date of |
|---|---|---|---|
| Cell Line | Number | Depositor | Deposition |
| NM-F9 | DSM ACC 2606 | Nemod | Aug. 14, 2003 |
| | | Biotherapeutics | |
| NM-D4 | DSM ACC 2605 | Nemod | Aug. 14, 2003 |
| | | Biotherapeutics | |
| NM-H9D8 | DSM ACC 2806 | Glycotope GmbH | Sep. 15, 2006 |
| NM-H9D8-E6 | DSM ACC 2807 | Glycotope GmbH | Oct. 5, 2006 |
| NM-H9D8- | DSM ACC 2856 | Glycotope GmbH | Aug. 8, 2007 |
| E6Q12 | | | |
| GT-2x | DSM ACC 2858 | Glycotope GmbH | Sep. 7, 2007 |
| GT-5s | DSM ACC 3078 | Glycotope GmbH | Jul. 28, 2010 |

1. An expression cassette comprising a promoter region and an expression element, wherein the expression element comprises a nucleic acid sequence coding for an extracellular region or a part thereof of a glycophorin protein, and wherein the expression cassette does not comprise a nucleic acid sequence coding for the entire glycophorin protein.

2. The expression cassette according to claim 1, which does not comprise a nucleic acid sequence encoding for a transmembrane region and/or a cytoplasmatic region of the glycophorin protein.

3. The expression cassette according to claim 12, wherein the glycophorin protein is a mammalian glycophorin protein.

4. The expression cassette according to claim 1, wherein the glycophorin protein is glycophorin A.

5. The expression cassette according to claim 1, wherein:
(a) the extracellular region of the glycophorin protein comprises amino acids 1 to 72 of SEQ ID NO: 1 or a homologue thereof;
(b) the part of the extracellular region of the glycophorin protein comprises at least 20 consecutive amino acids of amino acids 1 to 40 of SEQ ID NO: 1 or a homologue thereof; or
(c) the extracellular region or part thereof of the glycophorin protein comprises amino acids 2 to 38 of SEQ ID NO: 1.

6. (canceled)

7. (canceled)

8. The expression cassette according to claim 1, further comprising one or more of the elements selected from the group consisting of a transcription terminator region, a 5′ enhancer region, a 5′ untranslated region, a polyadenylation signal, a 3′ untranslated region and a 3′ enhancer region.

9. The expression cassette according to claim 1, comprising, in the direction of transcription, functionally linked to each other,
(i) a 5′ enhancer region,
(ii) a promoter region,
(iii) a 5′ untranslated region,
(iv) an expression element,
(v) a 3′ untranslated region,
(vi) a polyadenylation site,
(vii) a transcription terminator region, and
(viii) a 3′ enhancer region.

10. The expression cassette according to claim 1, wherein the expression element further comprises a nucleic acid sequence coding for a signal peptide.

11. The expression cassette according to claim 1, wherein the expression element further comprises a cloning site.

12. The expression cassette according to claim 1, wherein the expression element further comprises a nucleic acid sequence coding for a peptide of interest, wherein the peptide of interest and the extracellular region or part thereof of the glycophorin protein form a fusion peptide when expressing the expression element.

13. The expression cassette according to claim 12, wherein the peptide of interest is a glycoprotein or a part thereof.

14. The expression cassette according to claim 11, wherein the expression element further comprises a nucleic acid sequence coding for a protease recognition site, which is positioned between
(i) the nucleic acid sequence coding for the extracellular region or part thereof of the glycophorin protein and
(ii) the cloning site or the nucleic acid sequence coding for a peptide of interest;
wherein the extracellular region or part thereof of the glycophorin protein, the protease recognition site and the peptide of interest, if present, form a fusion peptide when expressing the expression element.

15. The expression cassette according to claim 14, wherein the nucleic acid sequences comprised in the expression element are functionally linked to each other in the direction of transcription in the following order:
(i) a nucleic acid sequence coding for a signal peptide;
(ii) the cloning site or a nucleic acid sequence coding for a peptide of interest;
(iii) the nucleic acid sequence coding for a protease recognition site;
(iv) the nucleic acid sequence coding for the extracellular region or part thereof of the glycophorin protein;
wherein the elements (i) to (iv), if present, form a fusion peptide when expressing the expression element.

16. The expression cassette according to claim 14, wherein the nucleic acid sequences comprised in the expression element are functionally linked to each other in the direction of transcription in the following order:
(i) a nucleic acid sequence coding for a signal peptide;
(ii) the nucleic acid sequence coding for the extracellular region or part thereof of the glycophorin protein.
(iii) the nucleic acid sequence coding for a protease recognition site;
(iv) the cloning site or a nucleic acid sequence coding for a peptide of interest;
wherein the elements (i) to (iv), if present, form a fusion peptide when expressing the expression element.

17. (canceled)

18. The expression cassette according to claim 1, which is adapted for expression in eukaryotic cells.

19. A vector comprising the expression cassette according to claim 1.

20. (canceled)

21. A host cell comprising the expression cassette according to claim 1.

22. A host cell comprising the vector according to claim 19.

23. The host cell according to claim 21, wherein the host cell is a eukaryotic cell.

24. A method for producing a peptide of interest, comprising the steps of
(a) providing a host cell according to claim 22, wherein the expression element in the host cell comprises a nucleic acid sequence coding for a peptide of interest;
(b) culturing the host cell under conditions at which the host cell expresses a fusion peptide comprising the peptide of interest and the extracellular region or part thereof of the glycophorin protein;
(c) isolating the peptide of interest; and
(d) obtaining the peptide of interest.

25. (canceled)

26. (canceled)

27. (canceled)

28. A fusion peptide comprising the extracellular region or a part thereof of a glycophorin protein and a peptide of interest, obtainable by the method according to claim 24.

29. A method for increasing the yield of a peptide of interest in recombinant production, comprising the step of expressing the peptide of interest as part of a fusion peptide which further comprises the extracellular region or a part thereof of a glycophorin protein.

30. (canceled)

31. The expression cassette according to claim 12, wherein the expression element further comprises a nucleic acid sequence coding for a protease recognition site, which is positioned between
(i) the nucleic acid sequence coding for the extracellular region or part thereof of the glycophorin protein and
(ii) a cloning site or the nucleic acid sequence coding for a peptide of interest;
wherein the extracellular region or part thereof of the glycophorin protein, the protease recognition site and the peptide of interest, if present, form a fusion peptide when expressing the expression element.


SEQUENCE LISTING (1 sequence)

SEQ ID NO: 1 - protein, 131 aa, Homo sapiens
  SOURCE 1..131: /mol_type=“protein” /note=“Glycophorin A” /organism=“Homo sapiens”
SSTTGVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ